(19) United States
(12) Patent Application Publication
(10) Pub. No.: US 2018/0092840 A1
(43) Pub. Date: Apr. 5, 2018

(54) ULTRASOUND-ENHANCED DRUG DELIVERY FOR TREATMENT OF ONYCHOMYCOSIS

(71) Applicant: The George Washington University, Washington, DC (US)

(72) Inventors: Vesna Zderic, Washington, DC (US); Alina Kline-Schoder, Hanover, NH (US); Zung Lee, Springfield, VA (US)

(21) Appl. No.: 15/670,519

(22) Filed: Aug. 7, 2017

Related U.S. Application Data
(60) Provisional application No. 62/404,020, filed on Oct. 4, 2016.

(51) Int. Cl. A61K 9/08; A61B 8/00; A61K 9/70

(52) U.S. Cl. CPC A61K 9/08; A61K 31/4178; A61K 9/7023; A61B 8/00

(57) ABSTRACT

A method of treating onychomycosis is provided. The method includes the steps of submerging a nail infected with onychomycosis in a solution containing at least one pharmaceutical agent, and applying ultrasound to the infected nail. Another method includes the steps of applying ultrasound to a subject's nail infected with onychomycosis first, and then applying a solution containing at least one pharmaceutical agent to the infected nail.

### CROSS-REFERENCE TO RELATED APPLICATION

This application claims priority under 35 U.S.C. § 119(e) to U.S. provisional patent application 62/404,020 filed on Oct. 4, 2016, which is hereby incorporated by reference in its entirety herein.

### FIELD OF THE INVENTION

The present invention relates to an improved drug delivery system and method for the treatment of onychomycosis. More particularly, the invention is directed to a method of delivering drugs to an infected nail through the application of ultrasound.

### BACKGROUND OF THE INVENTION

Millions of people suffer from onychomycosis, a fungal nail disorder characterized by thick, yellow, and painful nails. Onychomycosis is often overlooked and undertreated due to lack of appreciation on both the patient and physician side regarding the detrimental psychosocial effects it creates and the significant medical complications it can cause.¹ Onychomycotic patients not only have issues with normal nail functioning such as the wearing of shoes, and the trimming of nails but are also found to be less likely to form good relationships and to feel more socially excluded than those without the disease.^{2, 3} It has been found that the infection causes the most stigmatization, and overall significantly reduced physical, mental and social wellbeing in female and younger patients.⁴ It is estimated that as many as 32 million people in the United States alone are suffering from onychomycosis.¹ Onychomycosis is of particular concern for patients with diabetes. Diabetic patients who suffer from onychomycosis are at significantly higher risk of developing cellulitis, ulcers and gangerene.⁵

A common approach for onychomycosis treatment is an orally prescribed drug, Terbinafine. This drug, however, takes over six (6) months to work, has an overall failure rate of greater than 30%, and is associated with numerous side effects including elevated levels of liver enzymes and hepatitis.^{6, 7} Other treatment options include administration of an antifungal drug, Ciclopirox, which is applied in nail polish form. This treatment plan is often preferred because the drug has only non-serious, infrequently reported side effects. However, the cure rate for this drug is only 36% after six (6) months of daily application.^{8, 9} A more recently approved topical antifungal drug, Efinaconazole, has a slightly better cure rate, but has to be applied for up to ten (10) months and is relatively expensive compared to other treatments ($500 a month).¹⁰ The topical drugs have poor treatment effectiveness because they must permeate through the nail to reach the surface of the nail bed where the fungus resides. One of the most prominent reasons for poor nail permeability is the binding of the drug to the keratin in the nail, which decreases the amount of the drug that is able to travel through the entirety of the nail, and increases the amount of drug in the top few layers of the nail.¹¹

### SUMMARY OF THE INVENTION

Accordingly, the invention is directed to methods of improving the permeability of the nail so as to increase effectiveness of drug delivery to the infected nail.

It is an object of the invention to improve treatment of onychomycosis by applying ultrasound to a subject's nail in order to increase the amount of drug that permeates through the entirety of the nail allowing it to fully reach the treatment site.

In one aspect, a method of treating onychomycosis is provided. The method includes the steps of submerging a nail infected with onychomycosis in a solution containing at least one pharmaceutical agent, and applying ultrasound to the infected nail.

The invention is further directed to a method of treating onychomycosis including the steps of applying ultrasound to a subject's nail infected with onychomycosis, and applying a solution containing at least one pharmaceutical agent to the infected nail.

The invention also provides a system for the treatment of onychomycosis. The system includes a container for receiving a nail infected with onychomycosis, and an ultrasound device for applying ultrasound having a frequency of between 200 kHz and 3,000 kHz for a time of 1-10 minutes to the infected nail. The container is filled with a solution containing at least one pharmaceutical agent.

These and other objects of the invention, as well as many of the intended advantages thereof, will become more readily apparent when reference is made to the following description, taken in conjunction with the accompanying drawings.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 is a side view of the setup for a luminosity experiment according to an embodiment of the invention;

FIG. 2A-E are photographs of nails taken after completion of the luminosity experiment illustrated in FIG. 1;

FIG. 3 is a graph of the luminosity values of the tested nails at different ultrasound frequencies according to the experiment illustrated in FIG. 1;

FIG. 4 is a graph of the relative temperature increase in the vicinity of the tested nails measured at different ultrasound frequencies during the experiment illustrated in FIG. 1;

FIG. 5 is a side view of the setup for a diffusion cell experiment according to an embodiment of the invention;

FIGS. 6A-B are microscopic images of the control nail and nail exposed to 600 kHz from the experiment illustrated in FIG. 5;

FIG. 7 is a graph of the dilution of the dye measured at different ultrasound frequencies during the experimentation illustrated in FIG. 5;

FIG. 8 is a graph of the relative temperature increase in the vicinity of the tested nails measured at different ultrasound frequencies during the experiment illustrated in FIG. 5;

FIGS. 9A-B are side views of the PZFlex modeling setup used in connection with the experimentation of FIG. 5;

FIG. 10 is a graph of the temperature increase in a modeled human toe at different exposed ultrasound frequencies according to an embodiment of the invention; and

FIG. 11 is a graph of the dilution of the dye measured at different ultrasound frequencies during experimentation according to another embodiment of the invention.

### DETAILED DESCRIPTION

The invention relates to improved systems and methods of treating onychomycosis through the use of ultrasound technology. The application of ultrasound to the nail prior to administration of the drug is shown to increase delivery of the drug to the infected nail. The methods disclosed herein are shown to be safe for human application.

Ultrasound has been used successfully to increase drug delivery to the skin and eyes, but there is only preliminary evidence about its applicability to increase drug delivery to the nail bed.^{12, 13} Ultrasound application to the skin and eyes is used to treat different medical conditions, thus the treatment (drugs) would be different than those used to treat onychomycosis. Without being bound by any particular theory, it is believed that the main mechanism of ultrasonic action in ultrasound-enhanced drug delivery is cavitation-induced production of micrometer size pores in the surface barrier layer and streaming.^{13, 14} For example, inertial cavitation was shown to be the cause of alteration in barrier properties and structure of stratum corneum and increase in transdermal drug delivery.^{30, 31} Ultrasound also causes micro-streaming and bulk fluid streaming.¹⁵ Streaming is due to the reduction in strength of the ultrasound waves due to their absorption and scattering.¹⁶ Microstreaming is the flow of fluids due to the application of ultrasound. Microstreaming and bulk fluid streaming have been previously shown to facilitate drug delivery through the skin.¹⁷ A combination of streaming and cavitation are believed to be the mechanisms for increasing drug delivery through the nail.

The invention generally relates to a method of treating onychomycosis according to the following steps: (1) submerging a subject's nail infected with onychomycosis in a solution containing at least one pharmaceutical agent; and (2) applying ultrasound to the infected nail. The systems disclosed herein include the second step of applying ultrasound to create pores in the nail, such that the pharmaceutical agent is better able to penetrate the nail and the nail bed for improved treatment of the onychomycosis. Additionally, the streaming action of the ultrasound helps enhance the delivery of the pharmaceutical agent to the nail by “pushing” the agent through the pores created in the nail. In this embodiment, step (1) of submerging a subject's nail infected with onychomycosis in a solution containing at least one pharmaceutical agent, and step (2) of applying ultrasound to the infected nail may be done in sequence (step (1) performed before step (2)), or each of these steps may be performed simultaneously.

In another embodiment, the method of treating onychomycosis may be performed according to the following steps: (1) applying ultrasound to a subject's nail infected with onychomycosis; and (2) applying a solution containing at least one pharmaceutical agent to the infected nail.

In one embodiment, the ultrasound may be applied using focused or unfocused ultrasonic transducers depending on the needs of a particular application. In one embodiment, the nail is sonicated at frequencies of from about 200-1,200 kHz, preferably about 400-1,000 kHz, and most preferably from about 600-1,000 kHz. In another embodiment where focused ultrasound transducers are used, higher frequencies may be applied, such as, for example, 200 kHz to 3,000 kHz, preferably 400 kHz to 3,000 kHz. The intensity is preferably about 0.1 to 2 W/cm² at a duty cycle of about 10-100%. In a preferred embodiment, the intensity is about 0.1 to 1 W/cm² at a duty cycle of 100%.

The nail may be sonicated for about 1-10 minutes, preferably about 2-8 minutes, and most preferably about 3-6 minutes. The ultrasound may be applied at any distance suitable for a particular treatment. In one embodiment, the nail is sonicated at the optimal distance for unfocused ultrasound transducers, or “DFF,” as defined below.

Once the nail is sonicated, the pharmaceutical agent may then be administered as directed. The pharmaceutical agent may be selected from any known topical nail fungal drug, including, but not limited to, ciclopirox (e.g., Penlac®), terbinafine (e.g., Lamisil®), efinaconazole (e.g., Jublia®), and ketoconazole. In a preferred embodiment, these drugs are applied to the infected nail in a solution form. For example, the solution may contain 40% (by volume) ciclopirox in ethanol.

As set forth in the examples presented herein, the use of ultrasound was shown to increase nail permeability by up to 95% and the ultrasound parameters are shown to be safe to apply to the human toe. Depending on the ultrasound parameters and the particular pharmaceutical preparation being applied to the human toe nail, permeability could increase by at least 35% and up to about 20 times using the methods and systems disclosed herein.

The invention will now be described in conjunction with the following, non-limiting examples.

### Example 1

A first experiment was performed to determine the permeability of a dye into a nail in the presence of ultrasound frequencies at different parameters (hereinafter, the “luminosity test”). In this experiment, the dye was used to mimic a drug compound (that would be used to treat onychomycosis) so as to evaluate how the drug may be absorbed into a nail in the presence of ultrasound frequencies.

The drug mimicking compound used in the luminosity test was a standard food coloring water soluble blue dye (FD&C Blue No. 1—Brilliant Blue FCF, E133) with a molecular weight of 792.84 g/mol (commercially available from Flavors&Colors.com of Walnut, Calif., USA). Comparably, ciclopirox, the most commonly used topical drug for onychomycosis treatment, has a molecular weight of 207.27 g/mol. Both the blue dye and the nail drug are considered to be small molecules, and for small molecules, the permeability varies inversely with molecular weight, meaning that ciclopirox would be assumed to have a greater permeability than the blue dye used in the experiments.¹⁸ Additionally, both the drug-mimicking compound (the blue dye) and the drug are hydrophilic. Because the keratin of the nail is hydrophilic, the nail is permeable to hydrophilic substances such as the dye.¹⁹

The luminosity tests were carried out on porcine nails from pigs' feet (without onychomychosis) which were obtained from Sioux-Preme Packing Company (Sioux Center, Iowa, USA). The nails were separated from the feet using a scalpel and razor before being stored at 10.6° C. until their use. The pieces of porcine nail were cut to be approximately the size of a human nail (1 cm×1 cm×0.1 cm).

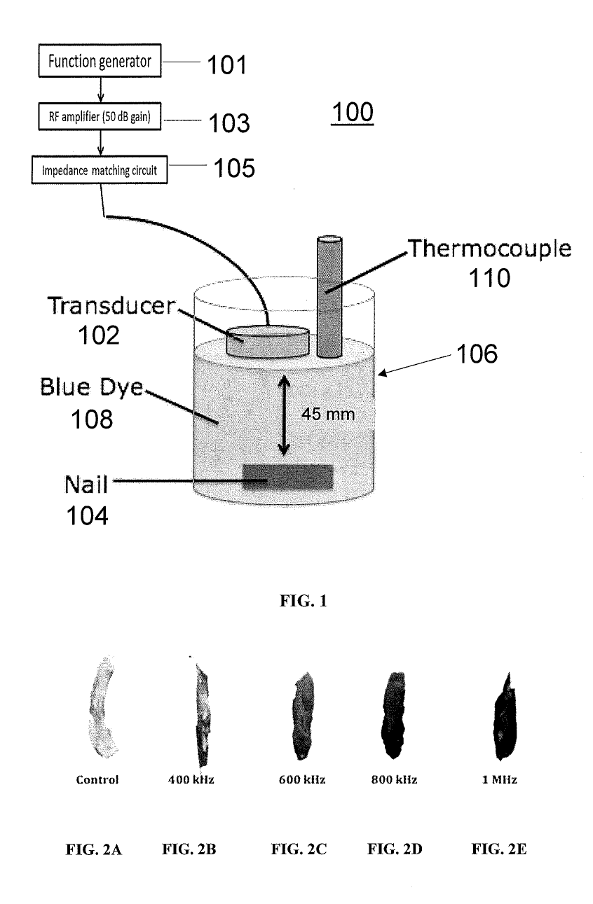

The nails were then subjected to ultrasound with the system 100 of FIG. 1. In this particularly luminosity test, unfocused circular ultrasonic transducers 102 were used (although focused ultrasonic transducers may also be used). The unfocused circular ultrasonic transducers 102 (commercially available from Sonic Concepts, Inc., Bothell, Wash., USA) had an active diameter of 15 mm and center frequencies of 400 kHz, 600 kHz, 800 kHz and 1 MHz. The ultrasonic waveforms were developed by a function generator 101 (commercially available from Agilent Technologies, Santa Clara, Calif., USA) and amplified to obtain a 10 db to 50 dB gain by an RF amplifier 103 (commercially available as 150A100B RF Amplifier from Amplifier Research Corporation, Souderton, Pa., USA). The impedance matching circuit 105 causes the ultrasound transducer load appear as 50Ω real impedance and 0Ω imaginary impedance for optimal transmission of the power from the amplifier 103.

The ultrasound used for the experiment was set to 1 W/cm² at the top and center of the nail at a 100% duty cycle. Each of the above-mentioned four frequencies was applied according to the discrete frequencies of the particular ultrasound transducer 102.

Ultrasound was applied to the nail 104 in a container for example a 50 mL beaker 106 in a water bath held at 37° C. (commercially available as Thermo Haake® DC10-P21 from Thermo Fisher Scientific Inc., Waltham, Mass., USA), as illustrated in FIG. 1. The beaker was filled with the previously-described blue dye 108, and the nail 104 was sonicated from a distance of about 45 mm for about five (5) minutes. A thermocouple 110 was placed near the nail to measure temperature throughout the treatment. Control nails were exposed to the dye for about five (5) minutes, but with no ultrasound sonication to serve as comparative examples. It will be noted that the container can be any suitable container for receiving one or more infected toes, and holds the solution having a pharmaceutical agent for treating the infection. The ultrasound can be applied directly to the toe, such as by touching the ultrasound to the toe, or via the solution. In addition, the ultrasound can be applied, before, after or during application of the solution to the toe.

Once the sonication was complete, the nails were rinsed and images of the nails facing upwards and downwards and their cross sections were taken. The images of the cross sections were analyzed using Photoshop (Photoshop™ 10.0 commercially available from Adobe Systems Incorporated of San Jose, Calif., USA) to compare the average brightness of each of the images, which correlates directly to the level of diffusion of the dye through the nails. On the scale used by Photoshop™, the brightness constant b was developed as follows: 250 represented a pure white picture and 0 represented a pure black picture. This number was adjusted to form a luminosity value ν that increases with an increase in diffusion as follows:

ν=10/b

The luminosity value ν was compared to the control experiments using an unequal variance two-tailed Student's t-test (n=−8).

The images of the nails are provided in FIGS. 2A-E. As can be seen, the change in nail color consistently increases as the level of ultrasound increases, with the highest level of ultrasound—1 MHz—exhibiting the greatest change in color in the nail (FIG. 2E). The Control nail, which was not exposed to any ultrasound, exhibited very little, if any, change in color (FIG. 2A).

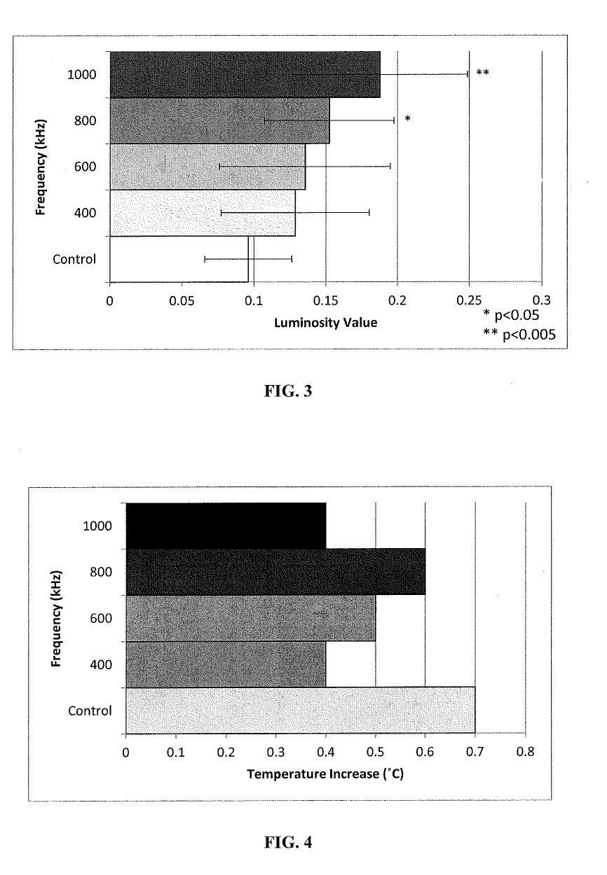

The luminosity values ν at each ultrasound frequency are provided in FIG. 3 as an average over eight tests. Generally, it was found that a higher frequency of ultrasound correlated to a higher luminosity constant, and therefore more diffusion of dye into the nail. The average luminosity value obtained for the Control nails which were not exposed to any ultrasound was 0.096. This value was much lower than the corresponding values for any of the nails exposed to ultrasound. The nails exposed to 400 kHz ultrasound had an average luminosity value of 0.129, whereas the nails exposed to 600 kHz ultrasound had an average value of 0.135. However, the average value for the nails exposed to 800 kHz ultrasound was 0.152 (corresponding to a 58.3% increase as compared to the Control nails, p<0.05), and the average value for the nails exposed to 1 MHz ultrasound was 0.187 (corresponding to a 94.8% increase as compared to the Control nails, p<0.005). Both the 800 kHz and 1 MHz tests were found to be statistically different from the Control value, indicating that more dye was permeating through the nails exposed to ultrasound as compared to the Control. Because the most compound delivery (i.e., dye absorption) was observed at higher frequencies, it is likely that the mechanisms of action in this case are bulk streaming and micro-streaming.¹⁶ Another possible mechanism for the increased drug delivery is cavitation. As set forth previously, cavitation is the production of micrometer size pores in the surface barrier layer.^{13, 14} Because most of the compound delivery through the nail appears to occur at higher frequencies, it is likely that cavitation does not have a critical impact on the delivery because its effect is greatest at lowest frequencies.

To determine the ultrasound intensity distribution in this luminosity test, and to execute the safety simulations, PZFlex modeling software (commercially available from Weidlinger Associates Inc., of New York, N.Y., USA) was used. PZFlex is a finite-element analysis software that utilizes an explicit time-domain to calculate the pressure and thermal effects of ultrasound. For all PZFlex simulations, a desktop computer (commercially available as a Dell® T5500 from Dell Inc. of Round Rock, Tex., USA) with 40 GB of memory and a dual-core 2.66 GHz Intel processor was used. Following PZFlex manufacturer recommendations in order to guarantee optimal spatial resolution, the grid size was made to be one fifteenth of the exposure wavelength for each applied ultrasound frequency. The convergence of this model was validated in previous studies at the same applied ultrasound parameters.²⁴ For each simulation, the axis was set to be symmetric and the boundary conditions were set to be absorbing. In each experiment the ultrasound was applied at frequencies of 400 kHz, 600 kHz, 800 kHz, and 1 MHz. The acoustical and thermal characteristics of the materials included in the simulations are indicated in Table 1 below.

TABLE 1
Tissue Properties used in PZFlex Simulations

| | Speed | | | |
|---|---|---|---|---|
| | of Sound | Specific Heat | Thermal Conductivity | Density |
| | (m/s) | (J/kgK) | (W/mK) | (kg/cm³) |
| Nail | 2549 | 1680 | 0.291 | 1270 |
| Skin | 1537 | 3391 | 0.293 | 1093 |
| Tendon | 1576 | 3432 | 0.449 | 1110 |
| Bone | 2405 | 2274 | 0.29 | 1330 |

To simulate the intensity, the following materials were used: nail, water to model the blue dye 108 (see FIG. 1), and glass to model the beaker 106. Water is an accurate model for the dye, because the dye has similar properties to water and is hydrophilic.²⁴ The luminosity simulation model used a constant distance—45 mm—from the transducer to the nail. For each frequency, the near field to far field transition distance (DFF) was calculated using the following equation, with D as the diameter of the transducer, f as the frequency, and V as the velocity of sound:

DFF=D²f/4V²⁵

The DFF distances calculated for 400 kHz, 600 kHz, 800 kHz, and 1 MHz are 10.5, 20.25, 30.0, and 30.75 mm respectively. At distances greater than the DFF, the wave propagation and intensity are predictable.²⁵ For this experiment, a distance greater than the DFF for all four frequencies (45 mm) was used, predicting consistent wave propagation. Ultrasound intensity was measured using radiation force balance as described in a previous study.²⁶ The intensity distribution in the specific experimental setups was further quantified using PZFlex simulations. A continuous ultrasonic beam was used with an exposure time of five (5) minutes at each of the aforementioned frequencies as utilized in the experiments. The results of these simulations are shown in Table 2 below, and all intensities were found to be 1+/−0.1 W/cm², which agreed with the radiation force balance measurement data.

TABLE 2
Intensity Results for Luminosity Experiment

| Frequency (kHz) | Luminosity Intensity (W/cm²) |
|---|---|
| 400 | 1.02 |
| 600 | 1.04 |
| 800 | 1.06 |
| 1000 | 0.96 |

To ascertain the safety of the application of ultrasound to a nail, the temperature of the dye near the nail was measured at each frequency and at every minute during the experimentation. A thermocouple 110 (commercially available as Wavetek Waterman TMD90 manufactured by Wavetek of San Diego, Calif., USA) having a range of 200-650° C., a resolution of 0.1° C., and an accuracy of 0.1% was used. The temperature results are provided in FIG. 4. The overall increase in temperature is shown at each applied frequency, with the baseline measurements being made at room temperature (23° C.). The temperature increases for all of the experiments were relatively constant, around 0.5° C. (with highest increase of 0.7° C. observed in the Control experiment), and are not expected to impact the dye uptake into the nail.

### Example 2

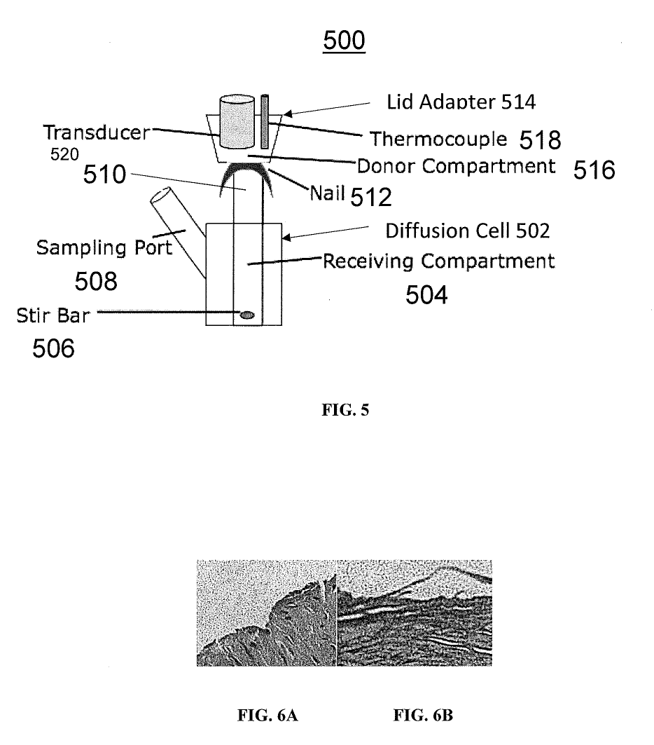

A second set of experiments was conducted utilizing a diffusion cell system 500, as set forth in FIG. 5 (hereinafter, the “diffusion cell test”). As described more fully below, a diffusion cell 502 having a receiving compartment 504 containing a stir bar 506 were positioned at the bottom of the system 500. A sampling port 508 projected from a side of the receiving compartment 504 to collect liquids contained therein. A nail adapter 510 fitted with a nail 512 was positioned above the diffusion cell 502. A lid adapter 514 was positioned above the nail 512. The lid adapter 514 contained a donor compartment 516, which held the blue dye, as well as a thermocouple 518 and a transducer 520. The parameters of the diffusion cell test were the same as set forth above with respect to Example 1, except in this example, spectrophotometry and a Franz Diffusion Cell were used to measure how much of the blue dye compound permeated all the way through the nail from a donor compartment (filled with the dye) to a receiver compartment, using dilution at different ultrasound parameters.

The diffusion cell (commercially available from PermeGear, Inc., Hellertown, Pa., USA) was fit with a custom-made nail adapter to avoid leakage of dye around the nail and a lid adapter so that 50 mL of dye and the transducer could easily fit above the nail adapter, as illustrated in FIG. 5. The nail adapter was made from plastic that screws together tightly and secures to the diffusion cell using small metal clamps. The lid adapter was made from a plastic beaker and secured onto the top of the nail adapter using epoxy. During the experimentation, the receiver compartment of the diffusion cell was filled with saline solution and a magnetic stirring bar which was spun at 450 RPM to ensure the compartment was fully mixed. The entire diffusion cell was placed in a water bath at 37° C. The donor compartment was then filled with 50 mL of the previously-described blue dye. Before being placed in the donor compartment, the blue dye was maintained at room temperature for at least 20 minutes. The transducer was placed 85 mm from the nail (as opposed to 45 mm from the nail in the luminosity experiment). The nail was then sonicated for five (5) minutes.

After sonication, the diffusion cell experiment continued for another 55 minutes to allow the dye to travel through the entirety of the nail. The Control nails were not treated with any ultrasound and were exposed to the dye solution for 60 minutes. The published literature for lag time in the nail is not very consistent. Some studies suggested that the lag time is as high as 400 hours, while other studies found a time closer to 15 minutes depending on the energy source supplied.^{20, 21} Because of these inconsistencies, 60 minutes of nail exposure to the dye solution was used for this diffusion cell test.

After the completion of the experiment, 2 mL of solution was collected from the receiving compartment of the diffusion cell and its absorption was measured with the spectrophotometer (commercially available as UVmini-1240 from Shimadzu Scientific Instruments of Columbia, Md., USA) using saline as a base. The wavelength used for measurement was found to be 630 nm by performing an initial calibration curve on the blue dye. This number was consistent with published values.²² Two serial dilutions totaling 26 measurements of dye in saline were also performed at this wavelength to develop an equation to convert from absorption measurement to dilution. Using an unequal variance Student's two-tailed t-test, ultrasound-treated groups were compared with control groups (n=6).

Next, two samples of nails subjected to each ultrasound frequency, including the Control nails, were fixed in formalin, cut to 5 μm slices, prepared using hematoxylin and eosin (H&E) and periodic acid Schiff (PAS), and fixed to a slide to conduct a histological study. The biopsies were observed under a microscope and documented as illustrated in FIGS. 6A-B. Specifically, the nail exposed to 600 kHz ultrasound and the Control nail were imaged by a microscope at 400×. As illustrated in FIGS. 6A-B, the histology results found no structural difference between the Control nail (FIG. 6A) and the ultrasound-treated nail (FIG. 6B). These images show that the Control nail and the ultrasound-treated nail have similar appearance, which indicates the safety of the ultrasound application.

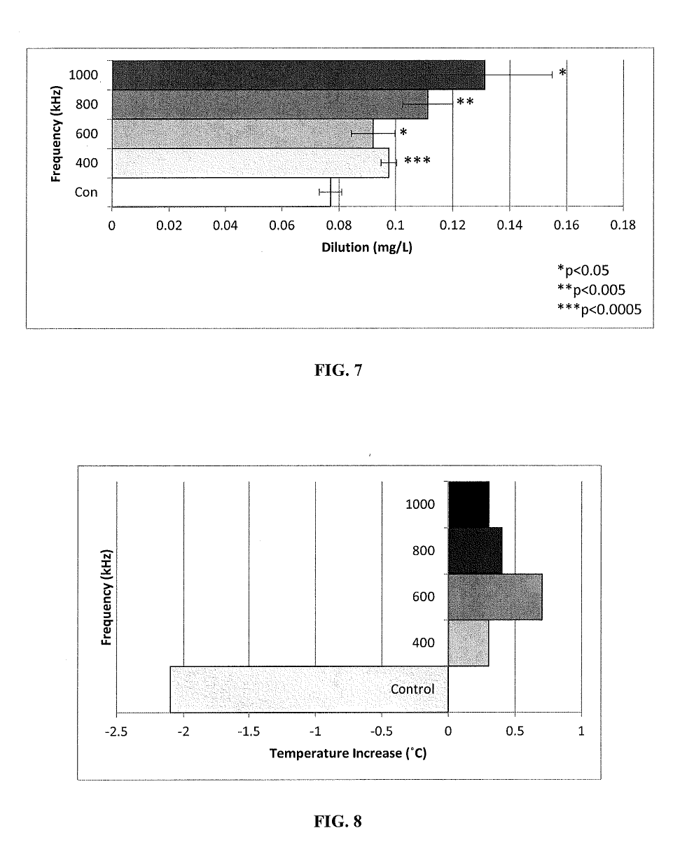

The comparison in permeability of the nails is shown in FIG. 7. Ultrasound application was found to increase nail permeability by 26.7% at 400 kHz, 19.5% at 600 kHz, 44.5% at 800 kHz and 70.3% at 1 MHz (n=6). All four of these frequencies were found to be statistically different (p<0.05), as compared to the Control values.

As in the luminosity test, temperature was also measured according to the same parameters set forth in Example 1. The temperature results are provided in FIG. 8. A maximum increase of 0.7° C. was found in the 600 kHz experiment, while the other frequencies exhibits less than 0.5° C. increase.

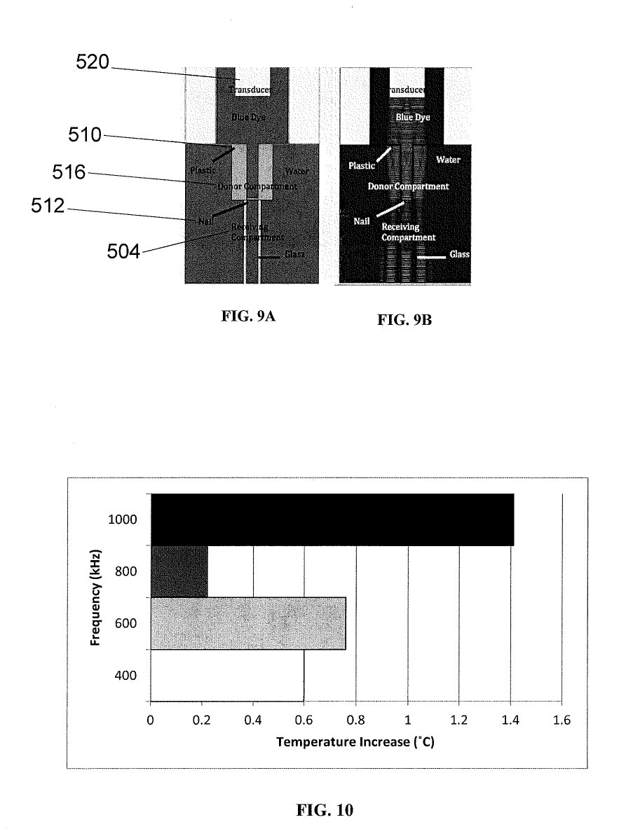

Lastly, in this diffusion cell test, the method to determine ultrasound intensity distribution and to execute safety simulations was the same as that set forth in Example 1 (using the PZFlex modeling software), with a few exceptions. First, to simulate the intensity in the diffusion cell experiments, glass was used to model the diffusion cell 502, plastic was used to model the two nail adaptors 510, and water was used to model the blue dye and the saline in the diffusion cell 502. This modeling setup is best illustrated in FIGS. 9A-9B. The left panel (FIG. 9A) shows the modeling setup that was built in the PZFlex modeling software, while the pressure effects of the ultrasonic application as simulated by PZFlex are shown in the right panel (FIG. 9B).

Second, the constant distance from the transducer to the nail was 85 mm (as opposed to 45 mm in the luminosity test). Ultrasound intensity was measured using radiation force balance as described in a previous study.²⁶ The intensity distribution in the specific experimental setups was further quantified using PZFlex simulations. A continuous ultrasonic beam was used with an exposure time of five (5) minutes at each of the aforementioned frequencies as utilized in the experiments. The results of these simulations are shown in Table 3 below, and all intensities were found to be 1+/−0.1 W/cm², which agreed with the radiation force balance measurement data. These intensity values are only slightly different from those in the luminosity test (see Table 2) due to the different reflection patterns in the different setup configurations and the different distances used between the nail and the ultrasound transducer.

TABLE 3
Intensity Results for Diffusion Cell Intensity

| Frequency (kHz) | Diffusion Cell Intensity (W/cm²) |
|---|---|
| 400 | 0.99 |
| 600 | 0.94 |
| 800 | 1.03 |
| 1000 | 0.99 |

### Example 3

A third experiment was conducted to further investigate the thermal safety of applying ultrasound to the human toe in a potential clinical application of this method for the treatment of onychomycosis. In this example, a two-dimensional, symmetrical computer model of the human toe was used. Because the human toe is already symmetrical, this is a realistic representation of the structure. In a real human toe, the toenail and other parts are slightly curved, but in this model all layers were modeled as straight and rectangular, which may have slightly increased simulated temperature values.²⁹ The human toe has two tendons: the lower tendon is the flexor hallucis longus, and the upper tendon is the extensor hallucis longus. These two tendons surround the phalanx. On each side of the tendons is skin, but the skin on the bottom is significantly thicker than the skin on the top of the phalanx. Finally, on top of the top skin is the nail. The thicknesses for each of these structures were estimated based upon known values³⁴⁻³⁸ and rounded to the nearest 0.5 mm, due to PZFlex allowances, as shown in Table 4 below.

TABLE 4
Thicknesses of Different Toe Structures

| Material | Thickness (mm) |
|---|---|
| Nail | 0.6 |
| Skin (top) | 0.2 |
| Upper tendon | 2.0 |
| Bone | 5.1 |
| Lower tendon | 2.0 |
| Skin (bottom) | 0.5 |

The substance surrounding the toe was modeled as water. Using the PZFlex software as described above in Example 1, the transducer was placed at the DFF distance which was calculated to be 10.5, 20.25, 30.0 and 30.75 cm for 400 kHz, 600 kHz, 800 kHz and 1 MHz, respectively. The applied intensity was 1 W/cm². The exposure times tested at each frequency were 15 seconds, 30 seconds, 1 minute, 2 minutes, 3 minutes, 4 minutes, and 5 minutes.

The thermal data is presented in FIG. 10. The maximum temperature increase when 400 kHz ultrasound was applied to the toe was 0.6° C. and occurred in the nail after five (5) minutes of exposure time. The maximum temperature increase when 600 kHz ultrasound was applied to the toe occurred after three minutes of exposure time and was about 0.8° C. in the bottom layer of the skin. The maximum temperature increase when 800 kHz ultrasound was applied to the toe occurred after three minutes of exposure and was 0.2° C. (measured in the nail). The maximum temperature increase when 1 MHz ultrasound was applied to the toe occurred after five (5) minutes of application and was about 1.4° C. (observed in the nail). In general, the highest temperature increases inside the human toe were found in the nail and in the bone and the lowest temperature increases were found in the tendons and in top and bottom layers of skin.

With respect to Examples 1-3, it is to be noted that the change in temperature due to the application of ultrasound is due to the balance of heat loss and heat gain in the exposed tissues.³⁰ The absorption characteristics of each tissue determines the heat gain, whereas the composition and vascularity of the tissue determines the heat loss. In the simulation, the majority of the maximum temperature increases occurred in the nail and in the bone. A larger absorption constant correlates to more heat absorption, and the nail and bone have the highest absorption coefficients of the tissues that compose the toe.³⁰ Therefore, it is logical that these are the two tissues in which the highest temperature increase occurs.

The results set forth herein generally showed a linear correlation between exposure time and increased temperature, however, many frequencies had several peak outliers with a high temperature increase at a low time interval. This is particularly noticeable in the 1 MHz tests. The linearity found is consistent with other studies performed, however, many of these tests also have outliers at lower time intervals, similar to the results set forth herein. For example, in a study performed by Draper et al. to assess the temperature change in muscle due to 1 MHz ultrasound at various intensities, the results were linear but also had some clear deviation from the normal. In the Draper study, the tests were performed every 15 seconds which provided a more visible trend.³¹

Using the European Committee for Medical Ultrasound's safety considerations, a temperature increase of 1.5° C. is considered safe. This indicates that all of the values found in the present experiments are expected to be safe, because the highest increase was only 1.2° C. There is some variation (3°) between the starting temperatures of temperature experiments. This is likely due to differences in the laboratory room temperature as the experiments were performed on different days, as well as the slight variations in initial temperature of the dye solution.

### Example 4

A fourth experiment was conducted similar to the parameters of Example 2, except that this experiment was conducted with an actual drug used to treat onychomycosis. All of the parameters in this Example 4 were the same as Example 2, including the use of pig feet, except for the following: the receiving compartment was filled with ethanol as opposed to saline solution, the sample in the receiving compartment was spun at 400 RPM, and the donor compartment was filled with 40% ciclopirox in ethanol (by volume).

The nails were loaded into the nail adapter as set forth in Example 2. The nails were then sonicated with the planar ultrasound transducer, which was placed about 45 mm above the nail, for five (5) minutes at an intensity of 1 W/cm² at a 100% duty cycle and at four frequencies: 400 kHz, 600 kHz, 800 kHz or 1 MHz. After the five (5) minutes of sonication, the experiment continued for another 55 minutes to allow the drug to travel through the entirety of the nail. The Control nails were not treated with any ultrasound and exposed to the drug solution for 60 minutes.

After the completion of the experiment, 2 mL of solution was collected from the receiving compartment of the diffusion cell and its absorption was measured with the spectrophotometer using ethanol as a base. The wavelength used for measurement was found to be 310 nm by performing an initial calibration curve on the ethanol. Two serial dilutions totaling 20 measurements of drug in ethanol were also performed to develop an equation to convert from absorption measurement to dilution. Using an unequal variance Student's Two-Tailed t-test, ultrasound-treated groups were compared with control groups (n=3).

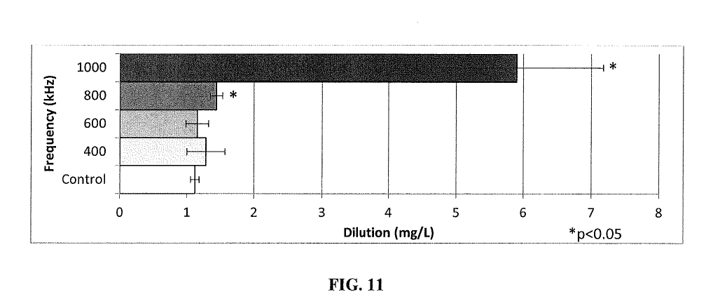

The comparison in permeability of the nails is shown in FIG. 11. It was found that a higher frequency of ultrasound correlated to a greater permeation of the nail. The tests conducted at 1 MHz and 800 kHz were statistically significant, resulting in an increase of 425% and 29% over the Control (p<0.05), respectively. The 600 kHz increase was 3.16% and the 400 kHz increase was 14.96%.

To be a fully complete model of the human toe, the nails used in the experiments would need perfusion. Although perfusion is recorded to affect the change in temperature due to ultrasound, it is reported to do so by decreasing the temperature by 10%, meaning that the results presented herein should be systematically 10% higher than those found in a living model.³² A previously published study that utilized vascular geometry to add perfusion to the tissue during tumor ablation demonstrates the possibility of utilizing this approach.³³

The following publications, as referenced throughout this disclosure, are incorporated herein by reference:
1. Scher R K, Tavakkol A, Sigurgeirsson B, et al. Onychomycosis: diagnosis and definition of cure. J Am Acad Dermatol 2007; 56(6):939-944.
2. Drake L A, Scher R K, Smith E B, et al. Effect of onychomycosis on quality of life. J Am Acad Dermatol 1998; 38(5):702-704.
3. Chan H H, Wong E T, Yeung C K. Psychosocial perception of adults with onychomycosis: a blinded, controlled comparison of 1,017 adult Hong Kong residents with or without onychomycosis. Biopsychosoc Med 2014; 1:8-15.
4. Szepietowski J C, Reich A. Stigmatisation in onychomycosis patients: a population-based study. Mycoses 2009; 52(4):343-349.
5. Roujeau J C, Sigurgeirsson B, Korting H C, Kerl H, Paul C. Chronic dermatomycoses of the foot as risk factors for acute bacterial cellulitis of the leg: a case-control study. Dermatology 2004; 209(4):301-307.
6. Baudraz-rosselet F, Rakosi T, Wili P B, Kenzelmann R. Treatment of onychomycosis with terbinafine. Br J Dermatol 1992; 39:40-46.
7. Amichai B, Mosckovitz R, Trau H, et al. Iontophoretic terbinafine HCL 10.0% delivery across porcine and human nails. Mycopathologia 2010; 169(5):343-349.
8. De Berker D. Fungal nail disease. N Engl J Med 2009; 360(20):2108-2116.
9. Gupta A K, Joseph W S. Ciclopirox 8% nail lacquer in the treatment of onychomycosis of the toenails in the United States. J Am Podiatr Med Assoc 2009; 90(10):495-501.
10. Elewski B E, Rich P, Poliak R, et al. Efinaconazole 10% solution in the treatment of toenail onychomycosis: Two phase III multicenter, randomized, double-blind studies. J Am Acad Dermatol 2013; 68(4):600-608.
11. Narasimha S, Wiskirchen D E, Bowers C P. Iontophoretic drug delivery across human nail. J Pharm Sci. 2007; 96(2):305-11.
12. Abadi D, Zderic V. Ultrasound-mediated nail drug delivery system. J Ultrasound Med 2011; 30(12):1723-1730.
13. Oberli M A, Schoellhammer C M, Langer R, Blankschtein D. Ultrasound-enhanced transdermal delivery: recent advances and future challenges. Ther Deliv 2014; 5(7):843-857.
14. Mo S, Coussios C C, Seymour L, Carlisle R. Ultrasound-enhanced drug delivery for cancer. Expert Opin Drug Deliv 2012; 9(12):1525-1538.
15. Cui J, Wei Y, Wang H. The study of low frequency ultrasound of enhance transdermal drug delivery. IEEE/ICME International Conference on Complex Medical Engineering 2007; 1221-1224.
16. Loh B, Hyun S, Ro P, Kleinstreuer C. Acoustic streaming induced by ultrasonic flexural vibrations and associated enhancement of convective heat transfer. J Acoust Soc Am 2002; 111(2):875-883.
17. Tang H, Mitragotri S, Blankschtein D, Langer R. Theoretical description of transdermal transport of hydrophilic permeants: Application to low-frequency sonophoresis. J Pharm Sci 2001; 90(5):545-568.
18. Basini P, Kr D B, Sudip D, Subhas S. Nail Drug Delivery System: A Review. J Adv Pharm Technol Res 2012; 2(3):101-109.
19. Mertin D, Lippold B C. In-vitro permeability of the human nail and of a keratin membrane from bovine hooves: penetration of chloramphenicol from lipophilic vehicles and a nail lacquer. J Pharm Pharmacol. 1997; 49(3):241-245.
20. Nair A B, Kim H D, Chakraborty B, et al. Ungual and trans-ungual iontophoretic delivery of terbinafine for the treatment of onychomycosis. J Pharm Sci 2009; 98(11):4130-4140.
21. Monti D, Saccomani L, Chetoni P, Burgalassi S, Saettone M F, Mailland F. In vitro transungual permeation of ciclopirox from a hydroxypropyl chitosan-based, water-soluble nail lacquer. Drug Dev Ind Pharm 2005; 31(1):11-17.
22. Orvis J, Orvis J, Koehler B. The Nature of Color Subtraction: A Guided Inquiry Experience. National Teachers Association 2007.
23. Paasch U, Mock A, Grunewald S, et al. Antifungal efficacy of lasers against dermatophytes and yeasts in vitro. Int J Hyperthermia 2013; 29(6):544-550.
24. Nabili M, Geist S, Zderic V. Thermal Safety of Ultrasound-enhanced Ocular Drug Delivery: A Modeling Study. Med Phys 2015; 42(10):5604-5615.
25. Christensen D A. Ultrasonic bio-instrumentation. Hoboken, N.J.: John Wiley and Sons; 1988.
26. Suarez Castellanos I, Jeremic A, Cohen J, Zderic V. Ultrasound stimulation of insulin release from pancreatic beta cells as potential novel treatment for type 2 diabetes. Ultrasound Med Biol 2017; 43(6):1210-1222.
27. Tang H, Wang C C, Blankschtein D, Langer R. An investigation of the role of cavitation in low-frequency ultrasound-mediated transdermal drug transport. Pharm Res 2002; 19:1160-1169.
28. Tezel A, Mitragotri S. Interactions of inertial cavitation bubbles with stratum corneum lipid bilayers during low frequency sonophoresis. Biophysics Journal 2003; 85:3502-3512.
29. Nell D M, Myers M R. Thermal effects generated by high-intensity focused ultrasound beams at normal incidence to a bone surface. J Acoust Soc Am. 2010; 127(1): 549-59.
30. Barnett S B, Rott H D, Ter haar G R, Ziskin M C, Maeda K. The sensitivity of biological tissue to ultrasound. Ultrasound Med Biol. 1997; 23(6):805-12.
31. Draper D O, Castel J C, Castel D. Rate of temperature increase in human muscle during 1 MHz and 3 MHz continuous ultrasound. J Orthop Sports Phys Ther. 1995; 22(4):142-50.
32. Horder M M, Barnett S B, Vella G J, Edwards M J, Wood A K. In vivo heating of the guinea-pig fetal brain by pulsed ultrasound and estimates of thermal index. Ultrasound Med Biol 1998; 24(9):1467-74.
33. Hariharan P, Chang I, Myers M R, Banerjee R K. Radio-Frequency Ablation in a Realistic Reconstructed Hepatic Tissue. J Biomech Eng 2006; 129(3):354-364.
34. Jemec, G B. Ultrasound structure of the human nail plate. Arch Dermatol 1989; 125(5):643-646.
35. Agache P G, Humbert P. Measuring the skin: Non-invasive investigations, physiology, normal constants. Springer: Berlin; 2004.
36. Dias D T, Steimacher A, Bento A C, Neto A M, Baesso M L. Thermal characterization in vitro of human nail: photoacoustic study of the aging process. Photochem Photobiol 2007; 83(5):1144-1148
37. Dias D T, Steimacher A, Bento A C, Neto A M, Baesso M L. Thermal characterization in vitro of human nail: photoacoustic study of the aging process. Photochem Photobiol 2007; 83(5):1144-1148.
38. Duck F A. Physical properties of tissues. Academic Press; 1990.

Accordingly, the foregoing description and drawings should be considered as illustrative only of the principles of the invention. The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

What is claimed is:

1. A method of treating onychomycosis, comprising the steps of:
(a) submerging a nail infected with onychomycosis in a solution containing at least one pharmaceutical agent; and
(b) applying ultrasound to the infected nail.

2. The method of claim 1, wherein the ultrasound has a frequency of between 200-1,200 kHz.

3. The method of claim 2, wherein the ultrasound has a frequency is between 400-1,000 kHz.

4. The method of claim 3, wherein the ultrasound has a frequency is between 800-1,000 kHz.

5. The method of claim 1, wherein the ultrasound has a frequency of between 200-3,000 kHz.

6. The method of claim 1, wherein the ultrasound is applied to the nail for about 1-10 minutes.

7. The method of claim 6, wherein the ultrasound is applied to the nail for about 3-6 minutes.

8. The method of claim 1, wherein the ultrasound has an intensity of about 0.1-2 W/cm² at a 10%-100% duty cycle.

9. The method of claim 1, wherein the nail permeability is increased by at least 35% and up to about 20 times.

10. The method of claim 1, wherein the pharmaceutical agent is at least one of ciclopirox, terbinafine, efinaconazole, and ketoconazole.

11. The method of claim 10, wherein the solution contains 40% ciclopirox and ethanol.

12. The method of claim 1, wherein the ultrasound is applied to the nail from a distance of about 2-100 mm.

13. The method of claim 1, wherein step (a) is performed before step (b).

14. The method of claim 1, wherein step (a) and step (b) are performed simultaneously.

15. A method of treating onychomycosis, comprising the steps of:
(a) applying ultrasound to a subject's nail infected with onychomycosis; and
(b) applying a solution containing at least one pharmaceutical agent to the infected nail.

16. The method of claim 15, wherein step (a) is performed before step (b).

17. The method of claim 15, wherein the step of applying a solution is done by submerging the nail in the solution.

18. A system for treatment of onychomycosis, comprising:
a container for receiving a subject's toe having a nail infected with onychomycosis, wherein the container contains a solution containing at least one pharmaceutical agent; and
an ultrasound device for applying ultrasound having a frequency of between 200 kHz and 3,000 kHz for a time of 1-10 minutes to the infected nail.

19. The system of claim 17, wherein the solution contains 40% ciclopirox and ethanol.

20. The system of claim 17, wherein the ultrasound has an intensity of about 0.1-2 W/cm² at a 10%-100% duty cycle.

21. The system of claim 17, wherein the ultrasound is applied to the nail from a distance of about 2-100 mm.
